(19) United States
(12) Patent Application Publication
(10) Pub. No.: US 2004/0167097 A1
(43) Pub. Date: Aug. 26, 2004

(54) EPO D + 5-FU/gemcitabine

(76) Inventors: Yiqing Zhou, Lafayette, CA (US); Robert G. Johnson, Lafayette, CA (US)

Correspondence Address: Gary W. Ashley, Kosan Biosciences, Inc., 3832 Bay Center Place, Hayward, CA, 94545, US

(21) Appl. No.: 10/683,203

(22) Filed: May 3, 2004

Related U.S. Application Data
(60) Provisional application No. 60/417,535, filed on Oct. 9, 2002.

(51) Int. Cl. A61K 31/7072; A61K 31/513; A61K 31/427

(52) U.S. Cl. 514/50; 514/365; 514/269

(57) ABSTRACT

Methods and compositions for treating hyperproliferative diseases using combinations of one or more epothilones and one or more nucleoside analogs. In some embodiments, the combination includes epothilone D and 5-fluorouracil or 5′-deoxy-5-fluoro-N-[(pentyloxy)carbonyl]-cytidine.

### CROSS-REFERENCE TO RELATED APPLICATIONS

This application claims priority under 35 U.S.C. 119(e) to U.S. Provisional Application Serial No. 60/417,535, filed 9 Oct. 2002, the contents of which are incorporated herein by reference in their entirety.

### FIELD OF THE INVENTION

The present invention relates to treatments for hyperproliferative diseases, such as cancer. More particularly, the present invention provides treatment modalities including the combination of an epothilone and a nucleoside analog, such as 5-fluorouracil. The present invention has applications in the fields of medicine and pharmacology.

### BACKGROUND

Epothilone D is among epothilones that can be isolated from mutated strains of Sorangium cellulosum, or from heterologous hosts expressing the epothilone polyketide synthase genes, and is known to bind to microtubules at the same site as paclitaxel. Epothilone D is 12,13-deoxyepothilone B; epothilone B is a major secondary metabolite of the S. cellulosum organism. Published reports have shown that epothilone D has dramatic antitumor effects in mice and is qualitatively markedly superior in this property to, for example, 15-azaepothilone B (Chou, et al. 2001). Methods to produce epothilone D using a genetically modified organism, as described in U.S. Ser. No. 09/560,367, filed 28 Apr. 2000, now allowed, and incorporated herein by reference, permit practical quantities of this compound to be produced to take advantage of its properties of stabilizing microtubules and inducing mitotic arrest. These important advances allow production of amounts of epothilone D sufficient for clinical use. Epothilone D, importantly, has antitumor efficacy both in paclitaxel-sensitive and paclitaxel-resistant cell lines and xenographs, although, as stated above, binding to microtubules by epothilone D is at the same site as binding by paclitaxel.

Experience with administration of paclitaxel has demonstrated that it is desirable to optimize regimens. Accordingly, the present invention is directed to particularly suitable protocols for epothilone administration to tumor patients in combination with other anticancer agents to provide a synergistically enhanced therapy.

### BRIEF DESCRIPTION OF THE DRAWINGS

FIG. 1A-FIG. 1C shows graphs of the Combination Index (“CI”) versus Effect for combinations of epothilone D and 5-FU on DLD-1 cells. FIG. 1A is a graph of CI versus Effect in which epothilone D and 5-FU are applied to the cells simultaneously. FIG. 1B is a graph of CI versus Effect for the combination in which DLD-1 cells are first incubated with epothilone D for 24 hours, 5-FU is applied to the cells, and the cells are incubated with the epothilone D-5-FU combination for 48 hours. FIG. 1C is a graph of CI versus Effect for the combination in which DLD-1 cells are first incubated with 5-FU for 24 hours, epothilone D is applied to the cells, and the cells are incubated with the epothilone D-5-FU combination for 48 hours.

FIG. 2A-FIG. 2C shows graphs of the Combination Index (“CI”) versus Effect for combinations of epothilone D and 5-FU on HCT-15 cells. FIG. 2A is a graph of CI versus Effect for the combination in which epothilone D and 5-FU are applied to the cells simultaneously. FIG. 2B is a graph of CI versus Effect for the combination in which HCT-15 cells are first incubated with epothilone D for 24 hours, 5-FU is applied to the cells, and the cells are incubated with the epothilone D-5-FU combination for 48 hours. FIG. 2C is a graph of CI versus Effect for the combination in which HCT-15 cells are first incubated with 5-FU for 24 hours, epothilone D is applied to the cells, and the cells are incubated with the epothilone D-5-FU combination for 48 hours.

FIG. 3A-FIG. 3C shows graphs of the Combination Index (“CI”) versus Effect for combinations of epothilone D and 5-FU on HCT-116 cells. FIG. 3A is a graph of CI versus Effect in which epothilone D and 5-FU are applied to the cells simultaneously. FIG. 3B is a graph of CI versus Effect for the combination in which HCT-116 cells are first incubated with epothilone D for 24 hours, 5-FU is applied to the cells, and the cells are incubated with the epothilone D-5-FU combination for 48 hours. FIG. 3C is a graph of CI versus Effect for the combination in which HCT-116 cells are first incubated with 5-FU for 24 hours, epothilone D is applied to the cells, and the cells are incubated with the epothilone D-5-FU combination for 48 hours.

### SUMMARY OF THE INVENTION

In one aspect, the invention provides methods for treating hyperproliferative disease, such as cancer, using a combination of an epothilone and a nucleoside analog. In some embodiments, the nucleoside analog used in the combination is effective to treat cancer alone or in combination with one or more drugs that are not epothilones. In more specific embodiments, the nucleoside analog is selected from the group consisting of: azacitidine, cladribine, cytarabine, floxuridine, fludarabine phosphate, 5-fluorouracil, gemcitabine, pentostatin, uracil mustard, and 5′-deoxy-5-fluoro-N-[(pentyloxy)carbonyl]-cytidine (sold under the trade name XELODA® (Roche)).

In another aspect, the invention provides methods for treating non-cancer diseases characterized by hyperproliferation.

In another aspect, the invention provides methods for treating disease comprising administering the combinations described herein in certain dosing regimens, also described herein. In certain embodiments, the epothilone and the nucleoside analog are administered simultaneously. In certain other embodiments, the epothilone and the nucleoside analog are administered sequentially. In certain embodiments, the epothilone is administered prior to administration of the nucleoside analog. In certain other embodiments, the epothilone is administered subsequent to administration of the nucleoside analog. In another aspect, the invention provides a combination of one or more epothilones and one or more nucleoside analogs for separate, simultaneous or sequential use in the treatment of a hyperproliferative disease. In another aspect, the invention provides for the use of one or more epothilones and one or more nucleoside analogs for the manufacture of a medicament for use in conjunction for the treatment of a hyperproliferative disease. In another aspect, the invention provides for the use of one or more epothilones for the manufacture of a medicament for administration in conjunction with one or more nucleoside analogs for the treatment of a hyperproliferative disease.

### DETAILED DESCRIPTION OF THE INVENTION

The present invention provides methods for treating hyperproliferative disease, such as cancer, using a combination of an epothilone and a nucleoside analog. In some embodiments, the nucleoside analog used in the combination is effective to treat cancer alone or in combination with one or more drugs that are not epothilones. In more specific embodiments, the nucleoside analog is selected from the group consisting of: azacitidine, cladribine, cytarabine, floxuridine, fludarabine phosphate, 5-fluorouracil, gemcitabine (2′,2′-difluorodeoxycytidine), pentostatin, uracil mustard, and 5′-deoxy-5-fluoro-N-[(pentyloxy)carbonyl]cytidine (sold under the trade name XELODA® (Roche)). These nucleoside analogs have a proven history of efficacy in the treatment of various cancers, as is well known in the art. As demonstrated below, epothilone D acts synergistically (i.e., the combined effect of the two drugs is greater than the sum of the effects of each drug individually) with a variety of nucleoside analogs in a variety of cell lines, suggesting methods for enhanced anticancer therapy against a range of cancer types.

The epothilone used in the pharmaceutical compositions of the invention can be any epothilone, and, more particularly, any epothilone having useful therapeutic properties (Hoefle, et al. 1993; Nicolaou, et al. 1998; Reichenbach, et al. 1998; Danishefsky, et al. 1999a; Danishefsky, et al. 1999b; Hoefle, et al. 1999; Nicolaou, et al. 1999a; Nicolaou, et al. 1999b; Vite, et al. 1999a; Vite, et al. 1999b; Vite, et al. 1999d; c; Hoefle, et al. 2000a; Hoefle, et al. 2000b; Danishefsky, et al. 2001a; Danishefsky, et al. 2001b; Santi, et al. 2001; Avery 2002; Danishefsky, et al. 2002; Nicolaou, et al. 2002a; Nicolaou, et al. 2002b; Wessjohann and Scheid 2002; White, et al. 2002). Such epothilones can be obtained using any combination of total chemical synthesis, partial chemical synthesis, or chemobiosynthesis methods and materials known to those of skill in organic chemistry, medicinal chemistry, and biotechnology arts (Hoefle, et al. 1993; Hoefle and Kiffe 1997; Hofle and Kiffe 1997; Schinzer, et al. 1997; 1998; Hofle and Sefkow 1998; Mulzer and Mantoulidis 1998; Nicolaou, et al. 1998; Reichenbach, et al. 1998; Schinzer, et al. 1998; Wessjohann and Gabriel 1998; Wessjohann and Kalesse 1998; Altmann, et al. 1999; Danishefsky, et al. 1999a; Danishefsky, et al. 1999b; Hoefle, et al. 1999; Hofmann, et al. 1999; Kim and Borzilleri 1999; Kim and Johnson 1999; Klar, et al. 1999a; b; Mulzer and Mantoulidis 1999; Nicolaou, et al. 1999a; Nicolaou, et al. 1999b; Schupp, et al. 1999; Vite, et al. 1999a; Vite, et al. 1999b; Vite, et al. 1999d; c; Beyer and Mueller 2000; Borzilleri, et al. 2000; Buchmann, et al. 2000; Cabral 2000; Georg, et al. 2000; Gustafsson and Betlach 2000; Hoefle, et al. 2000a; Hoefle, et al. 2000b; Hofle, et al. 2000; Julien, et al. 2000; Kim and Johnson 2000; Li, et al. 2000; Mulzer, et al. 2000; Arslanian, et al. 2001; Danishefsky, et al. 2001a; Danishefsky, et al. 2001b; Kim and Johnson 2001; Klar, et al. 2001; Kumar, et al. 2001; Lee 2001; Li, et al. 2001); (Mulzer and Martin 2001; Santi, et al. 2001; Strohhaecker 2001; Vite, et al. 2001; Avery 2002; Danishefsky, et al. 2002; Dimarco, et al. 2002; Hoefle and Glaser 2002; Julien, et al. 2002; Khosla and Pfeifer 2002; Koch and Loiseleur 2002; Kuesters and Unternaehrer 2002; Li, et al. 2002; Nicolaou, et al. 2002a; Nicolaou, et al. 2002b; Santi, et al. 2002a; Santi, et al. 2002b; Santi, et al. 2002c; Smith, et al. 2002; Wessjohann and Scheid 2002; Wessjohann, et al. 2002; White, et al. 2002). Specific examples of epothilones having useful therapeutic properties include, but are not limited to, epothilone A, epothilone B, epothilone C, epothilone D, 4-desmethylepothilone D, azaepothilone B, 21-aminoepothilone B, 9,10-dehydroepothilone D, 9,10-dehydro-26-trifluoro-epothilone D, 11-hydroxyepothilone D, 19-oxazolylepothilone D, 10,11-dehydro-epothilone D, and 19-oxazolyl-10,11-dehydro-epothilone D.

In some embodiments, the combination of the invention includes epothilone D and a nucleoside analog. In some embodiments, the nucleoside analog used in the combination is effective to treat cancer alone or in combination with one or more drugs that are not epothilones. In more specific embodiments, the nucleoside analog is selected from the group consisting of: azacitidine, cladribine, cytarabine, floxuridine, fludarabine phosphate, 5-fluorouracil, gemcitabine, pentostatin, uracil mustard, and 5′-deoxy-5-fluoro-N-[(pentyloxy)carbonyl]-cytidine (sold under the trade name XELODA® (Roche)). More particular embodiments include epothilone D in combination with either 5-fluorouracil or 5′-deoxy-5-fluoro-N-[(pentyloxy)carbonyl]-cytidine.

Therapeutic Applications of the Invention

The present invention also includes methods for treating diseases such as, but not limited to, hyperproliferative diseases, including: cancers of the head and neck which include tumors of the head, neck, nasal cavity, paranasal sinuses, nasopharynx, oral cavity, oropharynx, larynx, hypopharynx, salivary glands, and paragangliomas; cancers of the liver and biliary tree, particularly hepatocellular carcinoma; intestinal cancers, particularly colorectal cancer; treat ovarian cancer; small cell and non-small cell lung cancer; breast cancer sarcomas, such as fibrosarcoma, malignant fibrous histiocytoma, embryonal rhabdomysocarcoma, leiomysosarcoma, neurofibrosarcoma, osteosarcoma, synovial sarcoma, liposarcoma, and alveolar soft part sarcoma; neoplasms of the central nervous systems, particularly brain cancer; lymphomas such as Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma. Clinically, practice of the methods and use of compositions described herein will result in a reduction in the size or number of the cancerous growth and/or a reduction in associated symptoms (where applicable). Pathologically, practice of the method and use of compositions described herein will produce a pathologically relevant response, such as: inhibition of cancer cell proliferation, reduction in the size of the cancer or tumor, prevention of further metastasis, and inhibition of tumor angiogenesis. The method of treating such diseases comprises administering a therapeutically effective amount of an inventive combination to a subject. The method may be repeated as necessary.

The methods and compositions of the present invention can be used in combination therapies. In other words, the inventive compounds and compositions can be administered concurrently with, prior to, or subsequent to one or more other desired therapeutic or medical procedures. The particular combination of therapies and procedures in the combination regimen will take into account compatibility of the therapies and/or procedures and the desired therapeutic effect to be achieved. Thus, the compositions described herein can be combined with other treatment modalities, such as surgery and/or radiation. In some embodiments of the present invention, an agent or procedure is further included to mitigate potential side effects from the inventive compound or composition such as diarrhea, nausea and vomiting. Diarrhea may be treated with antidiarrheal agents such as opioids (e.g. codeine, diphenoxylate, difenoxin, and loeramide), bismuth subsalicylate, and octreotide. Nausea and vomiting may be treated with antiemetic agents such as dexamethasone, metoclopramide, diphenyhydramine, lorazepam, ondansetron, prochlorperazine, thiethylperazine, and dronabinol.

In another aspect of the present invention, non-cancer disorders that are characterized by cellular hyperproliferation are treated. Illustrative examples of such disorders include but are not limited to: atrophic gastritis, inflammatory hemolytic anemia, graft rejection, inflammatory neutropenia, bullous pemphigoid, coeliac disease, demyelinating neuropathies, dermatomyositis, inflammatory bowel disease (ulcerative colitis and Crohn's disease), multiple sclerosis, myocarditis, myositis, nasal polyps, chronic sinusitis, pemphigus vulgaris, primary glomerulonephritis, psoriasis, surgical adhesions, stenosis or restenosis, scleritis, scleroderma, eczema (including atopic dermatitis. irritant dermatitis, allergic dermatitis), periodontal disease (i.e., periodontitis), polycystic kidney disease, and type I diabetes. Other examples include vasculitis (e.g., Giant cell arteritis (temporal arteritis, Takayasu's arteritis), polyarteritis nodosa, allergic angiitis and granulomatosis (Churg-Strauss disease), polyangitis overlap syndrome, hypersensitivity vasculitis (Henoch-Schonlein purpura), serum sickness, drug-induced vasculitis, infectious vasculitis, neoplastic vasculitis, vasculitis associated with connective tissue disorders, vasculitis associated with congenital deficiencies of the complement system, Wegener's granulomatosis, Kawasaki's disease, vasculitis of the central nervous system, Buerger's disease and systemic sclerosis); gastrointestinal tract diseases (e.g., pancreatitis, Crohn's disease, ulcerative colitis, ulcerative proctitis, primary sclerosing cholangitis, benign strictures of any cause including ideopathic (e.g., strictures of bile ducts, esophagus, duodenum, small bowel or colon); respiratory tract diseases (e.g., asthma, hypersensitivity pneumonitis, asbestosis, silicosis and other forms of pneumoconiosis, chronic bronchitis and chronic obstructive airway disease); nasolacrimal duct diseases (e.g., strictures of all causes including ideopathic); and eustachean tube diseases (e.g., strictures of all causes including ideopathic).

Dosage Levels and Administration

In another aspect, the invention provides methods for treating disease comprising administering the combinations described above in certain dosing regimens, described herein. The epothilone can be administered simultaneously with one or more of the above-described nucleoside analogs. Alternatively, one or more nucleoside analogs can be administered prior to the administration of the epothilone. Conversely, the epothilone can be administered prior to administration of the nucleoside analog(s). In addition, for those embodiments in which the epothilone is administered separately from the nucleoside analog(s), the administration of the later drug(s) can be delayed to provide greater therapeutic effect of the combination therapy. Those having skill in the pharmacology and medicine arts will be familiar with concepts, methods, and materials suitable to determine the temporal factors in administering non-simultaneous therapies. Example of relevant factor may include, but are not limited to, the patient's circadian rhythm, cell cycle characteristics relevant to the disease being treated (e.g., tumor cell type), and the pharmacokinetic parameters of the drugs being used.

As used herein, the term “epothilone” refers to any naturally occurring epothilone or chemical analog or derivative therof, e.g. epothilone D, or an epothilone selected from the group consisting of: epothilone A, epothilone B, epothilone C, 4-desmethylepothilone D, azaepothilone B, 21-aminoepothilone B, 9,10-dehydroepothilone D, 9,10-dehydro-26-trifluoro-epothilone D, 11-hydroxyepothilone D, 19-oxazolylepothilone D, 10,11-dehydro-epothilone D, 19-oxazolyl-10,11-dehydro-epothilone D, 9,10-dehydroepothilone B or D, and 26-trifluoro-9,10-dehydroepothilone B or D. The dosages are to be administered to a subject suffering from cancer or a non-cancer disorder characterized by cellular proliferation, and are of the order from about 1 mg/m² to about 200 mg/m² which may be administered as a bolus (in any suitable route of administration, including oral or intravenous administration) or a continuous infusion (e.g., one hour, three hours, six hours, 24 hours, 48 hours or 72 hours) every week, every two weeks, or every three weeks as needed. It will be understood, however, that the specific dose level for any particular patient depends on a variety of factors. These factors include the activity of the specific compound employed; the age, body weight, general health, sex, and diet of the subject; the time and route of administration and the rate of excretion of the drug; whether a drug combination is employed in the treatment; and the severity of the condition being treated.

In another embodiment, the dosage levels are from about 10 mg/m² to about 150 mg/m², preferably from about 10 mg/m² to about 75 mg/m² and more preferably from about 15 mg/m² to about 50 mg/m² once every three weeks as needed and as tolerated. In another embodiment, the dosage levels are from about 1 mg/m² to about 150 mg/m², preferably from about 10 mg/m² to about 75 mg/m² and more preferably from about 25 mg/m² to about 50 mg/m² once every two weeks as needed and as tolerated. In another embodiment, the dosage levels are from about 1 mg/m² to about 100 mg/m², preferably from about 5 mg/m² to about 50 mg/m² and more preferably from about 10 mg/m² to about 25 mg/m² once every week as needed and as tolerated. In another embodiment, the dosage levels are from about 0.1 mg/m² to about 25 mg/m², preferably from about 0.5 mg/m² to about 15 mg/m² and more preferably from about 1 mg/m² to about 10 mg/m² once daily as needed and tolerated.

In order to ensure that toxic limits are not exceeded, side effects are monitored, including peripheral neuropathy, which may manifest itself as numbness in the limbs, dizziness, and the like. Monitoring should begin at some relevant time after infusion; in general, the lower the dosage, the longer the interval between treatment and monitoring. For example, at a dose level of 9 to 60 mg/m² per infusion monitoring will typically start at day 5 and continue to day 15; however, at higher dosages such as 90 to 120 mg/m², monitoring should begin the day after infusion is terminated. Other side effects may include nausea and vomiting, fatigue, rash, alopecia, and alteration in vital signs such as orthostatic hypotension. Myelosuppression should also be monitored although myelosuppression is generally not seen with this drug. Myelosuppression may manifest itself as anemia, neutropenia, thrombocytopenia, and the like.

In general, the pharmacokinetics are favorable. Pharmacokinetics are not dose-dependent and the dependence of AUC on dosage was linear from 9 to 150 mg/m². The half-life of epothilone D has a mean value of 9.6±2.2 hours and a volume of distribution (Vz) is 172±74 l, indicating good drug penetration. This is somewhat higher on average than the values for paclitaxel which are 140±70 l. These pharmacokinetic parameters do not change for a second infusion as compared to a first infusion.

The effectiveness of the drug may be monitored by measuring bundling of microtubules in interphase cells. This is considered reasonable indicator of effectiveness of microtubule stabilizing agents such as paclitaxel or an epothilone. The bundle formation can readily be measured by immunofluorescence or Western blotting. In a typical determination, whole blood is collected from patients and mononuclear cells (PBMC's) are isolated for evaluation of bundle formation. Substantial amounts of bundle formation are obtained when the dosage is as low as 18 mg/m² and this increases with dosage. At 120 mg/m² most of the microtublules are bundled.

### EXAMPLES

The following Examples merely illustrate certain aspects of the present invention to aid those of skill in the art in the art in practicing the invention and do not limit the scope of the invention in any manner.

### Example 1

### Demonstration of Synergy between Epothilone D and 5-fluorouracil

Cell Lines and Reagents

Cancer cell lines were obtained from the American Type Culture Collection (Manassas, Va.). The cells were maintained in RPMI medium with 10% fetal bovine serum. Epothilone D was obtained from the Department of Process Science at Kosan Biosciences, Inc (Hayward, Calif.). 5-Fluorouracil (“5-FU”) was purchased from Sigma. Each compound was dissolved in dimethylsulfoxide (“DMSO”) at a concentration of about ten millimolar (10 mM) for epothilone D and about fifty millimolar (50 mM) for 5FU. The solutions were stored at −20° C. until used.

Cell Viability Assay and Combination Effect Analysis

The cells described above were seeded in duplicate in 96-well microtiter plates at 5,000 cells per well, and the cells were allowed to attach to the wells overnight. Serial dilutions of each drug were added to the wells, and the cells were incubated for 72 hours. The IC₅₀ for each compound was determined using the CELLTITER 96 AQUEOUS ONE SOLUTION CELL PROLIFERATION ASSAY (Promega, Madison, Wis.), which correlates with the number of live cells.

For the drug combination assay, the cells were seeded in duplicate in 96-well plates (5,000 cells/well). After an overnight incubation, the cells were treated with either drug alone or a combination of the two drugs equivalent to the ratio of their IC₅₀ values. Three different treatment schedules were used. The first treatment schedule used simultaneous exposure to both drugs for 72 hours. For the second schedule, the cells were exposed to epothilone D for 24 hours, and then 5-FU was added to the cells; the cells were incubated for 48 hours. In the third treatment schedule, the cells were exposed to 5-FU alone for 24 hours followed by addition of epothilone D for 48 hours. The viability of the cells for each experiment was determined using the CELLTITER 96 AQUEOUS ONE SOLUTION CELL PROLIFERATION ASSAY (Promega, Madison, Wis.).

The data from each of the combination analyses was analyzed using CALCUSYN software (Biosoft, Cambridge, UK), which determined a combination index (“CI”) value for each

Results

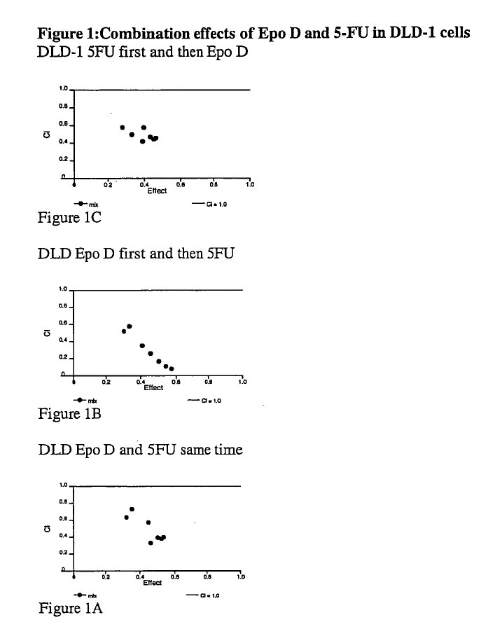

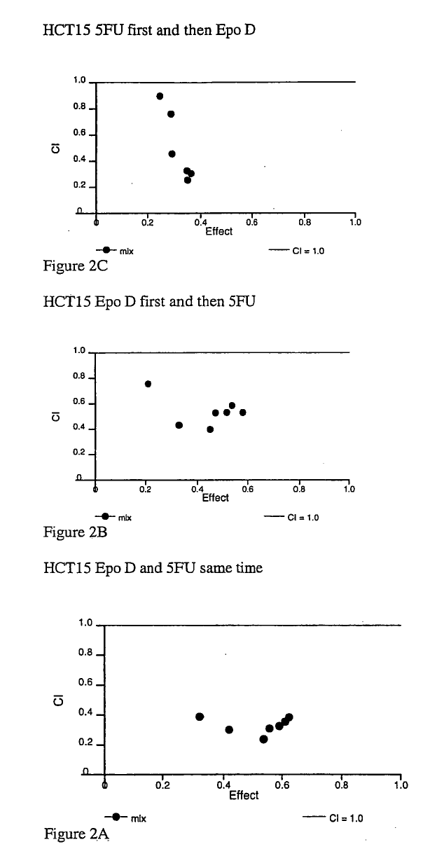

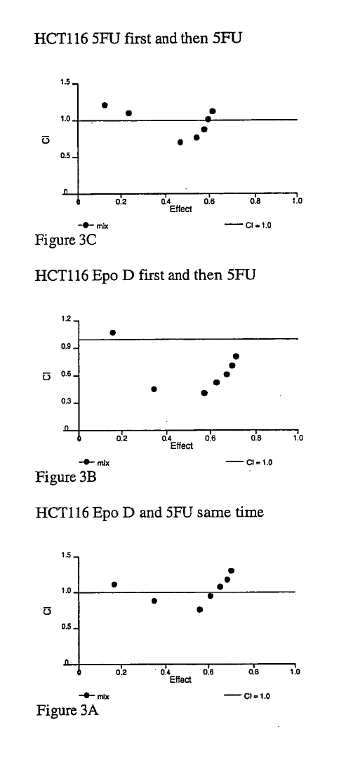

The data from each of the combination analyses was analyzed using CALCUSYN software (Biosoft, Cambridge, UK), which determined a combination index (“CI”) value for each combination in each of the three cell lines examined. A CI value less than one indicates the presence of a synergy between the two drugs; a CI value greater than one indicates an antagonism between the two drugs; and a CI value equal to one indicates an additive effect between the two drugs. The combination of epothilone D and 5-FU was determined to be synergistic for all cells lines tested, includingthe colon cancer cell lines DLD-1, HCT15, and HCT116, and the breast cancer cell lines AU565, MCF-7, MDA-MB-231, MX-1, T47D, and SKBr-3, as well as for all treatment schedules investigated (See FIGS. 1-3). This synergy was observed in the combination of epothilone D with both 5-fluorouracil and 5′-deoxy-5-fluorouridine. As 5′-deoxy-5-fluoro-N-[(pentyloxy)carbonyl]-cytidine is metabolized to 5′-deoxy-5-fluorouridine, which has been demonstrated to be synergistic with epothilone D, it is thus expected that 5′-deoxy-5-fluoro-N-[(pentyloxy)carbonyl]-cytidine will also be synergistic with epothilone D. Further, preliminary experiments have suggested that epothilone D can upregulate the production of thymidine phosphorylase in some tumor cells, the enzyme responsible for metabolism of 5′-deoxy-5-fluorouridine to 5-fluorouridine.

### BIBLIOGRAPHY

The following references are incorporated herein by reference in their entirety and for all purposes.

(1998). Ger. Offen. DE 19821954.

Altmann, K.-h., Bauer, A., et al. (1999). PCT Int. Appl. WO 9959985.

Arslanian, R. L., Ashley, G., et al. (2001). PCT Int. Appl. WO 0183800.

Avery, M. A. (2002). PCT Int. Appl. WO 0230356.

Beyer, S. and Mueller, R.-J. (2000). Ger. Offen. DE 19846493.

Borzilleri, R. M., Kim, S.-H., et al. (2000). PCT Int. Appl. WO 0057874.

Buchmann, B., Klar, U., et al. (2000). PCT Int. Appl. WO 0000485.

Cabral, F. (2000). PCT Int. Appl. WO 0071752.

Chou, T. C., O'Connor, O. A., et al. (2001). “The synthesis, discovery, and development of a highly promising class of microtubule stabilization agents: curative effects of desoxyepothilones B and F against human tumor xenografts in nude mice.” Proc Natl Acad Sci USA 98(14): 8113-8.

Danishefsky, S. J., Balog, A., et al. (1999a). PCT Int. Appl. WO 9943653.

Danishefsky, S. J., Balog, A., et al. (1999b). PCT Int. Appl. WO 9901124.

Danishefsky, S. J., Bertinato, P., et al. (2001a). U.S. Pat. No. 6,204,388.

Danishefsky, S. J., Lee, C. B., et al. (2001b). PCT Int. Appl. WO 0164650.

Danishefsky, S. J., Stachel, S. J., et al. (2002). U.S. patent application Publ.20020058286.

Dimarco, J. D., Gougoutas, J. Z., et al. (2002). PCT Int. Appl. WO 0214323.

Georg, G. I., Nair, S. K., et al. (2000). PCT Int. Appl. WO 0058254.

Gustafsson, C. and Betlach, M. C. (2000). U.S. Pat. No. 6,090,601.

Hoefle, G., Bedorf, N., et al. (1993). Ger. Offen. DE 4138042.

Hoefle, G. and Glaser, N. (2002). PCT Int. Appl. WO 0224712.

Hoefle, G., Glaser, N., et al. (2000a). Ger. Offen. DE 19907588.

Hoefle, G., Glaser, N., et al. (2000b). PCT Int. Appl. WO 0050423.

Hoefle, G. and Kiffe, M. (1997). Ger. Offen. DE 19542986.

Hoefle, G., Reichenbach, H., et al. (1999). PCT Int. Appl. WO 9965913.

Hofle, G., Glaser, N., et al. (2000). Eur. Pat. Appl. Ep 987268.

Hofle, G. and Kiffe, M. (1997). PCT Int. Appl. WO 9719086.

Hofle, G. and Sefkow, M. (1998). PCT Int. Appl. WO 9838192.

Hofmann, H., Mahnke, M., et al. (1999). PCT Int. Appl. WO 9942602.

Julien, B., Katz, L., et al. (2002). U.S. Pat. No. 6,410,301.

Julien, B., Katz, L., et al. (2000). PCT Int. Appl. WO 0031247.

Khosla, C. and Pfeifer, B. (2002). PCT Int. Appl. WO 0268613.

Kim, S.-H. and Borzilleri, R. M. (1999). PCT Int. Appl. WO 9927890.

Kim, S.-H. and Johnson, J. A. (1999). PCT Int. Appl. WO 9928324.

Kim, S.-H. and Johnson, J. A. (2000). PCT Int. Appl. WO 0071521.

Kim, S.-h. and Johnson, J. A. (2001). U.S. Pat. No. 6,320,045.

Klar, U., Gay, J., et al. (2001). PCT Int. Appl. WO 0166154.

Klar, U., Schwede, W., et al. (1999a). Ger. Offen. DE 19735575.

Klar, U., Schwede, W., et al. (1999b). Ger. Offen. DE 19735574.

Koch, G. and Loiseleur, O. (2002). PCT Int. Appl. WO 0257251.

Kuesters, E. and Unternaehrer, H. (2002). PCT Int. Appl. WO 0246196.

Kumar, A. M., Klein, J. P., et al. (2001). PCT Int. Appl. WO 0126693.

Lee, F. Y. (2001). PCT Int. Appl. WO 0172721.

Li, W., Matson, J. A., et al. (2000). PCT Int. Appl. WO 0039276.

Li, W.-s., Thornton, J. E., et al. (2002). PCT Int. Appl. WO 0260904.

Li, W. S., Thornton, J. E., et al. (2001). PCT Int. Appl. WO 0170716.

Mulzer, J. and Mantoulidis, A. (1998). Ger. Offen. DE 19726627.

Mulzer, J. and Mantoulidis, A. (1999). PCT Int. Appl. WO 9903848.

Mulzer, J., Mantoulidis, A., et al. (2000). Ger. Offen. DE 19848306.

Mulzer, J. and Martin, H. (2001). PCT Int. Appl. WO 0107439.

Nicolaou, C. K., He, Y., et al. (1998). PCT Int. Appl. WO 9825929.

Nicolaou, K. C., He, Y., et al. (2002a). U.S. Pat. No. 6,441,186.

Nicolaou, K. C., Hepworth, D., et al. (1999a). PCT Int. Appl. WO 9967253.

Nicolaou, K. C., King, N. P., et al. (2002b). U.S. Pat. No. 6,380,394.

Nicolaou, K. C., King, N. P., et al. (1999b). PCT Int. Appl. WO 9967252.

Reichenbach, H., Hofle, G., et al. (1998). PCT Int. Appl. WO 9822461.

Santi, D., Ashley, G., et al. (2002a). U.S. patent applicatioin Publ.20020052028.

Santi, D., Fardis, M., et al. (2001). PCT Int. Appl. WO 0192255.

Santi, D., Metcalf, B., et al. (2002b). PCT Int. Appl. WO 0208440.

Santi, D. V., Ashley, G., et al. (2002c). PCT Int. Appl. WO 0212534.

Schinzer, D., Limberg, A., et al. (1997). Ger. DE 19636343.

Schinzer, D., Limberg, A., et al. (1998). PCT Int. Appl. WO 9808849.

Schupp, T., Ligon, J. M., et al. (1999). PCT Int. Appl. WO 9966028.

Smith, A. B., Beauchamp, T. J., et al. (2002). U.S. patent application Publ.20020103387.

Strohhaecker, J. (2001). PCT Int. Appl. WO 0160976.

Vite, G. D., Borzilleri, R. M., et al. (1999a). PCT Int. Appl. WO 9954330.

Vite, G. D., Borzilleri, R. M., et al. (1999b). PCT Int. Appl. WO 9902514.

Vite, G. D., Kim, S.-H., et al. (2001). PCT Int. Appl. WO 0173103.

Vite, G. D., Kim, S.-H. K., et al. (1999c). PCT Int. Appl. WO 9954318.

Vite, G. D., Kim, S.-H. K., et al. (1999d). PCT Int. Appl. WO 9954319.

Wessjohann, L. A. and Gabriel, T. (1998). Ger. Offen. DE 19701758.

Wessjohann, L. A. and Kalesse, M. (1998). Ger. Offen. DE 19713970.

Wessjohann, L. A. and Scheid, G. (2002). Ger. Offen. DE 10051136.

Wessjohann, L. A., Scheid, G., et al. (2002). PCT Int. Appl. WO 0232844.

White, J. D., Carter, R. G., et al. (2002). U.S. patent application Publ.20020062030.

What is claimed is:

1. A method for treating hyperproliferative disease, said method comprising administering to a patient in need of such treatment a combination of one or more epothilones and one or more nucleoside analogs.

2. The method of claim 1 wherein administration of one or more epothilones and administration of one or more nucleoside analogs are simultaneous.

3. The method of claim 2 wherein the epothilone is selected from the group consisting of epothilone B, epothilone D, 21-hydroxyepothilone B, 21-hydroxyepothilone D, 21-aminoepothilone B, 21-aminoepothilone D, azaepothilone B, azaepothilone D, 9,10-dehydroepothilone B, 9,10-dehydroepothilone D, 26-trifluoro-9,10-dehydroepothilone B, and 26-trifluoro-9,10-dehydroepothilone D.

4. The method of claim 2 wherein the nucleoside analog is selected from the group consisting of azacitidine, cladribine, cytarabine, floxuridine, fludarabine phosphate, 5-fluorouracil, gemcitabine, pentostatin, uracil mustard, and 5′-deoxy-5-fluoro-N-[(pentyloxy)carbonyl]-cytidine.

5. The method of claim 2 wherein the epothilone is epothilone D and the nucleoside analog is selected from the group consisting of 5-fluorouracil and 5′-deoxy-5-fluoro-N-[(pentyloxy)carbonyl]-cytidine.

6. The method of claim 2 wherein administration of the epothilone results in a dosage of between about 1 mg/m² and about 200 mg/m².

7. The method of claim 1 wherein administration of one or more epothilone occurs first, followed by administration of one or more nucleoside analog.

8. The method of claim 7 wherein the epothilone is selected from the group consisting of epothilone B, epothilone D, 21-hydroxyepothilone B, 21-hydroxyepothilone D, 21-aminoepothilone B, 21-aminoepothilone D, azaepothilone B, azaepothilone D, 9,10-dehydroepothilone B, 9,10-dehydroepothilone D, 26-trifluoro-9,10-dehydroepothilone B, and 26-trifluoro-9,10-dehydroepothilone D.

9. The method of claim 7 wherein the nucleoside analog is selected from the group consisting of azacitidine, cladribine, cytarabine, floxuridine, fludarabine phosphate, 5-fluorouracil, gemcitabine, pentostatin, uracil mustard, and 5′-deoxy-5-fluoro-N-[(pentyloxy)carbonyl]-cytidine.

10. The method of claim 7 wherein the epothilone is epothilone D and the nucleoside analog is selected from the group consisting of 5-fluorouracil and 5′-deoxy-5-fluoro-N-[(pentyloxy)carbonyl]-cytidine.

11. The method of claim 7 wherein administration of the epothilone results in a dosage of between about 1 mg/m² and about 200 mg/m².

12. The method of claim 1 wherein administration of one or more nucleoside analog occurs first, followed by administration of one or more epothilone.

13. The method of claim 12 wherein the epothilone is selected from the group consisting of epothilone B, epothilone D, 21-hydroxyepothilone B, 21-hydroxyepothilone D, 21-aminoepothilone B, 21-aminoepothilone D, azaepothilone B, azaepothilone D, 9,10-dehydroepothilone B, 9,10-dehydroepothilone D, 26-trifluoro-9,10-dehydroepothilone B, and 26-trifluoro-9,10-dehydroepothilone D.

14. The method of claim 12 wherein the nucleoside analog is selected from the group consisting of azacitidine, cladribine, cytarabine, floxuridine, fludarabine phosphate, 5-fluorouracil, gemcitabine, pentostatin, uracil mustard, and 5′-deoxy-5-fluoro-N-[(pentyloxy)carbonyl]-cytidine.

15. The method of claim 12 wherein the epothilone is epothilone D and the nucleoside analog is selected from the group consisting of 5-fluorouracil and 5′-deoxy-5-fluoro-N-[(pentyloxy)carbonyl]-cytidine.

16. The method of claim 12 wherein administration of the epothilone results in a dosage of between about 1 mg/m² and about 200 mg/m².

17. The method of claim 12 wherein administration of the epothilone results in a dosage of between about 1 mg/m² and about 200 mg/m².

18. The method of claim 1 wherein said hyperproliferative disease is cancer.

19. The method of claim 18 wherein the cancer is selected from the group consisting of colorectal cancer, breast cancer, and non-small cell lung cancer.

20. The method of claim 19 wherein the cancer is colorectal cancer or breast cancer.

21. A combination of one or more epothilones and one or more nucleoside analogs for separate, simultaneous or sequential use in the treatment of a hyperproliferative disease.

22. The combination of claim 21, wherein the epothilone is selected from the group consisting of epothilone B, epothilone D, 21-hydroxyepothilone B, 21-hydroxyepothilone D, 21-aminoepothilone B, 21-aminoepothilone D, azaepothilone B, azaepothilone D, 9,10-dehydroepothilone B, 9,10-dehydroepothilone D, 26-trifluoro-9,10-dehydroepothilone B, and 26-trifluoro-9,10-dehydroepothilone D.

23. The combination of claim 21, wherein the nucleoside analog is selected from the group consisting of azacitidine, cladribine, cytarabine, floxuridine, fludarabine phosphate, 5-fluorouracil, gemcitabine, pentostatin, uracil mustard, and 5′-deoxy-5-fluoro-N-[(pentyloxy)carbonyl]-cytidine.

24. The combination of claim 21, wherein the epothilone is epothilone D and the nucleoside analog is selected from the group consisting of 5-fluorouracil and 5′-deoxy-5-fluoro-N-[(pentyloxy)carbonyl]-cytidine.

25. The combination of claim 21, wherein the hyperproliferative disease is cancer.

26. The combination of claim 25, wherein the cancer is selected from the group consisting of colorectal cancer, breast cancer, and non-small cell lung cancer.

27. The combination of claim 21, wherein the treatment involves administering the one or more epothilones and the one or more nucleoside analogs simultaneously.

28. The combination of claim 21, wherein the treatment involves administering the one or more epothilones first, followed by the one or more nucleoside analogs.

29. The combination of claim 21, wherein the treatment results in a dosage of the one or more epothilones of between about 1 mg/m² and about 200 mg/m².

30. Use of one or more epothilones and one or more nucleoside analogs for the manufacture of a medicament for use in conjunction for the treatment of a hyperproliferative disease.

31. The use of claim 30, wherein the epothilone is selected from the group consisting of epothilone B, epothilone D, 21-hydroxyepothilone B, 21-hydroxyepothilone D, 21-aminoepothilone B, 21-aminoepothilone D, azaepothilone B, azaepothilone D, 9,10-dehydroepothilone B, 9,10-dehydroepothilone D, 26-trifluoro-9,10-dehydroepothilone B, and 26-trifluoro-9,10-dehydroepothilone D.

32. The use of claim 30, wherein the nucleoside analog is selected from the group consisting of azacitidine, cladribine, cytarabine, floxuridine, fludarabine phosphate, 5-fluorouracil, gemcitabine, pentostatin, uracil mustard, and 5′-deoxy-5-fluoro-N-[(pentyloxy)carbonyl]-cytidine.

33. The use of claim 30, wherein the epothilone is epothilone D and the nucleoside analog is selected from the group consisting of 5-fluorouracil and 5′-deoxy-5-fluoro-N-[(pentyloxy)carbonyl]-cytidine.

34. The use of claim 30, wherein the hyperproliferative disease is cancer.

35. The use of claim 34, wherein the cancer is selected from the group consisting of colorectal cancer, breast cancer, and non-small cell lung cancer.

36. The use of claim 30, wherein the treatment involves administering the one or more epothilones and the one or more nucleoside analogs simultaneously.

37. The use of claim 30, wherein the treatment involves administering the one or more epothilones first, followed by the one or more nucleoside analogs.

38. The use of claim 30, wherein the treatment results in a dosage of the one or more epothilones of between about 1 mg/m² and about 200 mg/m².

39. Use of one or more epothilones for the manufacture of a medicament for administration in conjunction with one or more nucleoside analogs for the treatment of a hyperproliferative disease.

40. The use of claim 39, wherein the epothilone is selected from the group consisting of epothilone B, epothilone D, 21-hydroxyepothilone B, 21-hydroxyepothilone D, 21-aminoepothilone B, 21-aminoepothilone D, azaepothilone B, azaepothilone D, 9,10-dehydroepothilone B, 9,10-dehydroepothilone D, 26-trifluoro-9,10-dehydroepothilone B, and 26-trifluoro-9,10-dehydroepothilone D.

41. The use of claim 39, wherein the nucleoside analog is selected from the group consisting of azacitidine, cladribine, cytarabine, floxuridine, fludarabine phosphate, 5-fluorouracil, gemcitabine, pentostatin, uracil mustard, and 5′-deoxy-5-fluoro-N-[(pentyloxy)carbonyl]-cytidine.

42. The use of claim 39, wherein the epothilone is epothilone D and the nucleoside analog is selected from the group consisting of 5-fluorouracil and 5′-deoxy-5-fluoro-N-[(pentyloxy)carbonyl]-cytidine.

43. The use of claim 39, wherein the hyperproliferative disease is cancer.

44. The use of claim 43, wherein the cancer is selected from the group consisting of colorectal cancer, breast cancer, and non-small cell lung cancer.

45. The use of claim 39, wherein the treatment involves administering the one or more epothilones and the one or more nucleoside analogs simultaneously.

46. The use of claim 39, wherein the treatment involves administering the one or more epothilones first, followed by the one or more nucleoside analogs.

47. The use of claim 39, wherein the treatment results in a dosage of the one or more epothilones of between about 1 mg/m² and about 200 mg/m².
